(19) United States
(12) Patent Application Publication
(10) Pub. No.: US 2004/0175344 A1
(43) Pub. Date: Sep. 9, 2004

(54) Silicone-based moisture absorbing matrix, particularly for caring for wounds and/or for the pharmaceutical/cosmetic treatment of skin

(76) Inventor: Karl-Heinz Woller, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/472,872

(22) PCT Filed: Mar. 22, 2002
(86) PCT No.: PCT/EP02/03227

(30) Foreign Application Priority Data
Mar. 23, 2001 (DE) 101 14 382.6

(51) Int. Cl. A61K 7/06; A61K 7/11; A61K 35/78

(52) U.S. Cl. 424/70.12; 424/74; 424/745; 424/747; 424/764; 424/769; 424/778

(57) ABSTRACT

The invention relates to a silicone-based moisture absorbing matrix, particularly for caring for wounds and/or for the pharmaceutical/cosmetic treatment of skin, whereby the sticky matrix is comprised of: a) silicone; b) a gelling agent, and; c) optionally, a silicone resin.

The invention relates to a silicone-based moisture-absorbing matrix, in particular for wound care and/or pharmaceutical/cosmetic skin treatment.

Products comprising silicone are distinguished by very good skin compatibility and are widespread in medical technology. For example, catheters, electrode holders, tubing, implants and wound dressings are made of silicone. Adhesive products comprising silicone can at the same time have a very high adhesive force and despite this can be removed again very easily and without pain. A further advantage is the reduced scar formation of injuries which are covered with a silicone wound dressing.

It turns out to be disadvantageous that silicone is water repellent and cannot absorb any wound exudation at all. The principle of moist wound treatment can therefore not be carried out using wound dressings comprising silicone.

Transdermal therapeutic systems (TTS) for the delivery of active compounds through the skin have been known for a long time.

The topical application of medicaments by means of active compound-containing patch systems offers two main advantages:

Firstly, by means of this administration form release kinetics of the active compound of first order are realized, whereby a constant active compound level in the body can be maintained over a very long period of time.

Secondly, by means of the absorption route through the skin, the gastrointestinal tract and the first liver passage are avoided. As a result, selected pharmaceuticals can be administered effectively in a low dose. This is advantageous in particular if a local action of the pharmaceutical with circumvention of a systemic action is desired. This is the case, for example, in the treatment of rheumatic joint symptoms or myositis.

An embodiment of such transdermal systems which is well described in the specialist literature are matrix systems or monolithic systems in which the pharmaceutical is incorporated directly into the pressure-sensitive adhesive. Such an adhesive, active compound-containing matrix is equipped in the ready-to-use product on the one side with a carrier which is impermeable for the active compound, on the opposite side there is a carrier film equipped with a separating layer which is removed before application to the skin (kleben&dichten, No. 42, 1998, pp. 26 to 30).

A basic requirement for a TTS is a very good adhesion to the skin, which must be maintained over the entire period of time of the intended active compound dose. A frequently observed side effect, however, is the occurrence of skin irritation, which occurs particularly on relatively long or repeated application of a TTS to a constant body region. It is mainly caused by the constituents of the adhesive matrix. Painful removal of the active compound-containing patch again after a relatively long period of wearing is frequently also observed.

Repeated and long-lasting applications of adhesive systems to regions of the human body which are always the same are especially to be encountered in the field of stoma care. Hydrocolloids have been employed as an adhesive here for a long period and with great success. These in principle consist of a hydrophilic adhesive polymer matrix based on synthetic rubber, in which hydrophilic fillers based on, for example, alginates, cellulose or pectins which are insoluble in this matrix are present in disperse form.

In the development of hydrocolloids for stoma care, however, the adhesive properties to moist skin and the capability for liquid absorption are predominant.

EP 0 186 019 A1 describes an invention of an active compound patch using fillers which are swellable in water. Here, however, the positive influence of the organic filler on the release rate of the active compound is described. The filler component according to the invention is restricted to 30% by weight. The aspect of the reduction of skin irritation is not discussed. Additionally, the systems described are produced using adhesive resins.

It is the aim of the present invention to make available a silicone-based moisture-absorbing matrix, in particular for wound care and/or pharmaceutical/cosmetic skin treatment. In addition, this should be capable of the controlled delivery of a pharmaceutical, for example from the group consisting of the ethereal oils.

If the moisture-absorbing matrix is equipped to be self-adhesive, the side effects of a contact adhesive for transdermal systems mentioned—skin irritation and painful removal again—should be avoided, which results in a marked increase in wearer comfort for the patient.

This object is achieved by a moisture-absorbing matrix according to the main claim. The subject of the subclaims are advantageous embodiments of the matrix according to the invention.

Accordingly, the invention relates to a silicone-based moisture-absorbing matrix, in particular for wound care and/or pharmaceutical/cosmetic skin treatment, the adhesive matrix consisting of

a) silicone

b) gel-forming agent

c) optionally a silicone resin.

In a first advantageous embodiment of the invention, the matrix has the following composition:

| | |
|---|---|
| a) silicone: | 55 to 80% by weight, in particular 60 to 75% by |
| | weight |
| b) gel-forming agent: | 20 to 40% by weight, in particular 25 to 40% by |
| | weight |

The inorganic gel-forming agent(s) or thickener(s) can be advantageously chosen, for example, from the group consisting of the modified or unmodified, naturally occurring or synthetic layered silicates.

Although it is perfectly convenient to employ pure components, it is also possible, however, in an advantageous manner to incorporate mixtures of various modified and/or unmodified layered silicates into the compositions according to the invention.

Layered silicates, which are also called phyllosilicates, are to be understood in the context of this application as meaning silicates and aluminosilicates in which the silicate or aluminate units are linked to one another by means of three Si—O or Al—O bonds and form a corrugated sheet or layered structure. The fourth Si—O or Al—O valence is neutralized by cations. Between the individual layers exist weaker electrostatic interactions, for example hydrogen bonds. The layered structure, however, is mainly formed by strong, covalent bonds.

The stoichiometry of the sheet silicates is

(Si₂O₅²⁻) for pure silicate structures and

(AlₘSi²⁻ₘO₅ (^{3+m})⁻) for aluminosilicates.

m is a number greater than zero and less than 2.

If not pure silicates but aluminosilicates are present, the factor is to be taken into account that each Si⁴⁺ group replaced by Al³⁺ requires a further singly charged cation for charge neutralization.

The charge balance is preferably equalized by H⁺, alkali metal or alkaline earth metal ions. Aluminum as a counterion is also known and advantageous. In contrast to the aluminosilicates, these compounds are called aluminum silicates. “Aluminum aluminosilicates”, in which aluminum is present both in the silicate network, and as a counterion, are known and optionally advantageous for the present invention.

Layered silicates are well documented in the literature, for example in the “Lehrbuch der Anorganischen Chemie” [Textbook of Inorganic Chemistry], A. F. Hollemann, E. Wiberg and N. Wiberg, 91 st-100 th ed., Walter de Gruyter—Verlag 1985, passim, and “Lehrbuch der Anorganischen Chemie”, H. Remy, 12th ed., Akademische Verlagsgesellschaft, Leipzig 1965, passim. The layered structure of montmorillonite can be inferred from Römpps Chemie-Lexikon [Römpp's Chemical Encyclopedia] Franckh'sche Verlagshandlung W. Keller & Co., Stuttgart, 8th ed., 1985, p. 2668 ff.

Examples of layered silicates are:

| | |
|---|---|
| Montmorillonite | Na_{0.33}((Al_{1.67}Mg_{0.33})(OH)₂(Si₄O₁₀)) |
| often simplified: | Al₂O₃.4SiO₂.H₂O.nH₂O or Al₂[(OH)₂/Si₄O₁₀].nH₂O |
| kaolinite | Al₂(OH)₄(Si₂O₅) |
| illite | (K, H₃O)_{y}(Mg₃(OH)₂(Si_{4-y}Al_{y}O₁₀)) |
| and | (K, H₃O)_{y}(Al₂(OH)₂(Si_{4-y}Al_{y}O₁₀)) |
| | where y = 0.7-0.9 |
| beidellite | (Ca, Na)_{0.3}(Al₂(OH)₂(Al_{0.5}Si_{3.5}O₁₀)) |
| nontronite | Na_{0.33}(Fe₂(OH)₂(Al_{0.33}S_{13.67}O₁₀)) |
| saponite | (Ca, Na)_{0.33}((Mg, Fe)₃(OH)₂(Al_{0.33}S_{13.67}O₁₀)) |
| hectorite | Na_{0.33}((Mg, Li)₃(OH, F)₂(Si₄O₁₀)) |

Montmorillonite is the main mineral of naturally occurring bentonites.

Very advantageous inorganic gel-forming agents within the meaning of the present invention are aluminum silicates such as the montmorillonites (bentonites, hectorites and their derivatives such as quaternium-18 bentonite, quaternium-18 hectorite, stearalkonium bentonites or stearalkonium hectorites) or else magnesium aluminum silicates (Veegum® types) and sodium magnesium silicates (Laponite® types).

Montmorillonites are clay minerals belonging to the dioctahedral smectites and are materials which swell in water, but do not become plastic. The layer packets in the three-layered structure of the montmorillonites can be swollen by reversible intercalation of water (in a 2-7 fold amount) and other substances such as, for example, alcohols, glycols, pyridine, α-picoline, ammonium compounds, hydroxyaluminosilicate ions etc.

The chemical formula indicated above is only approximate; since M. possesses a great ion-exchange ability, Al can be exchanged for Mg, Fe²⁺, Fe³⁺, Zn, Pb (for example from pollutants in waste waters) Cr, also Cu and others. The negative charge of the octahedral layers resulting therefrom is equalized by means of cations, in particular Na⁺ (sodium montmorillonite) and Ca²⁺ (calcium montmorillonite is only very poorly swellable) in intermediate layer positions.

Within the meaning of the present invention, advantageous synthetic magnesium silicates or bentonites are marketed, for example, by Süd-Chemie under the trade name Optigel®.

An aluminum silicate which is advantageous within the meaning of the present invention is marketed, for example, by the R. T. Vanderbilt Comp., Inc., under the trade name Veegum®. The various Veegum® types, which are all advantageous according to the invention, are distinguished by the following compositions

| | (regular grade) | HV | K | HS | S-728 |
|---|---|---|---|---|---|
| SiO₂ | 55.5 | 56.9 | 64.7 | 69.0 | 65.3 |
| MgO | 13.0 | 13.0 | 5.4 | 2.9 | 3.3 |
| Al₂O₃ | 8.9 | 10.3 | 14.8 | 14.7 | 17.0 |
| Fe₂O₃ | 1.0 | 0.8 | 1.5 | 1.8 | 0.7 |
| CaO | 2.0 | 2.0 | 1.1 | 1.3 | 1.3 |
| Na₂O | 2.1 | 2.8 | 2.2 | 2.2 | 3.8 |
| K₂O | 1.3 | 1.3 | 1.9 | 0.4 | 0.2 |
| loss on | 11.1 | 12.6 | 7.6 | 5.5 | 7.5 |
| incineration | | | | | |

These products swell in water with the formation of viscous gels, which have an alkaline reaction. By organophilization of montmorillonite or bentonites (exchange of the intermediate layer cations for quaternary alkylammonium ions), products result (bentones) which are preferably employed for dispersion in organic solvents and oils, fats, ointments, dyes, lacquers and in detergents.

Bentone® is a trade name for various neutral and chemically inert gelling agents which are constructed from long-chain, organic ammonium salts and special types of montmorillonite. Bentones swell in organic media and cause these to swell. The gels are stable in dilute acids and alkalies, but on relatively long stirring with strong acids and alkalies they partially lose their gelling properties. On account of their organophilic character, the bentones are only wettable by water with difficulty.

The following Bentone® types are marketed, for example, by Kronos Titan: Bentone® 27, an organically modified montmorillonite, Bentone® 34 (dimethyldioctyl-ammonium bentonite), which is prepared according to U.S. Pat. No. 2,531,427 and swells better in lipophilic medium than in water because of its lipophilic groups, Bentone® 38, an organically modified montmorillonite, a cream-colored to white powder, Bentone® LT, a purified clay mineral, Bentone® gel MIO, an organically modified montmorillonite which is supplied suspended in mineral oil (SUS-71) in very fine form (10% bentonite, 86.7% mineral oil and 3.3% wetting agent), Bentone® gel IPM, an organically modified bentonite which is suspended in isopropyl myristate (10% bentonite, 86.7% isopropyl myristate, 3.3% wetting agent), Bentone® gel CAO, an organically modified montmorillonite which is taken up in castor oil (10% bentonite, 86.7% castor oil, 3.3% wetting agent), Bentone® gel Lantrol, an organically modified montmorillonite which is intended for further processing in paste form, in particular for the production of cosmetic compositions; 10% bentonite, 64.9 Lantrol (wool wax oil), 22.0 isopropyl myristate, 3.0 wetting agent and 0.1 propyl p-hydroxybenzoate, Bentone® gel Lan I, a 10% strength Bentone® 27 paste in a mixture of wool wax USP and isopropyl palmitate, Bentone® gel Lan II, a bentonite paste in pure, liquid wool wax, Bentone® gel NV, a 15% strength Bentone® 27 paste in dibutyl phthalate, Bentone® gel OMS, a bentonite paste in Shellsol T. Bentone® gel OMS 25, a bentonite paste in isoparaffinic hydrocarbons (Idopar® H), Bentone® gel IPP, a bentonite paste in isopropyl palmitate.

“Hydrocolloid” is the technological abbreviation for the name “hydrophilic colloid”, which is more correct per se. Hydrocolloids are macromolecules which have a mainly linear shape and have intermolecular interactive forces which make possible secondary and main valence bonds between the individual molecules and thus the development of a network-like structure. They are partially water-soluble natural or synthetic polymers which form gels or viscous solutions in aqueous systems. They increase the viscosity of the water by either binding water molecules (hydration) or else absorbing and enveloping the water in their interwoven macromolecules, simultaneously restricting the mobility of the water. Such water-soluble polymers are a large group of chemically very different natural and synthetic polymers whose common feature is their solubility in water or aqueous media. The prerequisite for this is that these polymers have a number of hydrophilic groups adequate for water solubility and are not too highly crosslinked. The hydrophilic groups can be of nonionic, anionic or cationic nature, for example as follows:

| | | | |
|---|---|---|---|
| 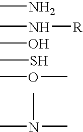 | 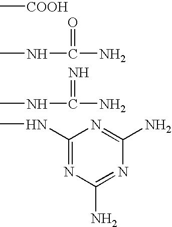 | 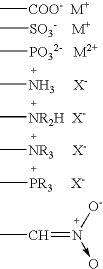 | 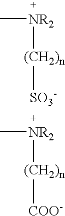 |

The group consisting of the cosmetically and dermatologically relevant hydrocolloids can be divided as follows into:

organic, natural compounds, such as, for example, agar-agar, carrageenin, tragacanth, gum arabic, alginates, pectins, polyoses, guar gum, carob bean flour, starch, dextrins, gelatin, casein,

organic, modified natural substances, such as, for example, carboxymethylcellulose and other cellulose ethers, hydroxyethyl- and -propylcellulose and microcrystalline cellulose the like,

organic, fully synthetic compounds, such as, for example, polyacrylic and polymethacrylic compounds, vinyl polymers, polycarboxylic acids, polyethers, polyimines, polyamides, polyurethanes

inorganic compounds, such as, for example, polysilicic acids, clay minerals such as montmorillonites, zeolites, silicic acids.

Microcrystalline cellulose is an advantageous hydrocolloid within the meaning of the present invention. It is obtainable, for example, from the “FMC Corporation Food and Pharmaceutical Products” under the trade name Avicel®. A particularly advantageous product within the meaning of the present invention is the type Avicel® RC-591, which is a modified microcrystalline cellulose which is composed to 89% of microcrystalline cellulose and to 11% of sodium carboxymethyl cellulose. Further commercial products of this class of raw material are Avicel® RC/CL, Avicel® CE-15, Avicel® 500.

Further advantageous hydrocolloids according to the invention are, for example, methylcelluloses, as which the methyl ethers of cellulose are named. They are distinguished by the following structural formula

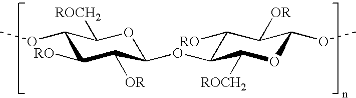

in which R can be a hydrogen or a methyl group.

Particularly advantageous within the meaning of the present invention are the cellulose mixed ethers, which are in general likewise named as methylcelluloses, which in addition to a dominant content of methyl groups, additionally contain 2-hydroxyethyl, 2-hydroxypropyl or 2-hydroxybutyl groups. (Hydroxypropyl)methyl celluloses are particularly preferred, for example those obtainable under the trade name Methocel® E4M from the Dow Chemical Comp.

According to the invention, sodium carboxymethylcellulose, the sodium salt of the glycolic acid ether of cellulose, for which R in structural formula I can be a hydrogen and/or CH₂—COONa, is furthermore advantageous. The sodium carboxymethylcellulose obtainable under the trade name Natrosol Plus 330 CS from Aqualon, also named cellulose gum, are particularly preferred.

Further preferred within the meaning of the present invention is xanthan (CAS No. 11138-66-2), also called xanthan gum, which is an anionic heteropolysaccharide which as a rule is formed by fermentation of corn sugar and is isolated as the potassium salt. It is produced from Xanthomonas campestris and some other species under aerobic conditions with a molecular weight of 2×10⁶ to 24×10⁶. Xanthan is formed of a chain containing β-1,4-bonded glucose (cellulose) with side chains. The structure of the subgroups consists of glucose, mannose, glucuronic acid, acetate and pyruvate. Xanthan is the name for the first microbial anionic heteropolysaccharide. It is produced by Xanthomonas campestris and some other species under aerobic conditions with a molecular weight of 2-15 10⁶. Xanthan is formed from a chain containing β-1,4-bonded glucose (cellulose) with side chains. The structure of the subgroups consists of glucose, mannose, glucuronic acid, acetate and pyruvate. The number of the pyruvate units determines the viscosity of the xanthan. Xanthan is produced in two-day batch cultures with a yield of 70-90%, based on carbohydrate employed. In this process, yields of 25-30 g/l are achieved. The work-up takes place after destroying the culture by precipitation with, for example, 2-propanol. Xanthan is then dried and ground.

An advantageous gel-forming agent within the meaning of the present invention is furthermore carrageen, an extract from north Atlantic red algae belonging to the Florideae (Chondrus crispus and Gigartina stellata) which forms gels and is constructed similarly to agar.

Frequently, the name carrageen is used for the dried algal product and carrageenan for the extract of this. The carrageen precipitated from the hot water extract of the algae is a colorless to sand-colored powder having a molecular weight range of 100,000-800,000 and a sulfate content of about 25%. Carrageen, which is very slightly soluble in warm water; on cooling forms a thixotropic gel, even if the water content is 95-98%. The solidity of the gel is brought about by the double helix structure of the carrageen. In the case of carrageenan, three main constituents are distinguished: the gel-forming κ-fraction consists of D-galactose-4-sulfate and 3,6-anhydro-α-D-galactose, which are alternately glycosidically bonded in the 1,3- and 1,4-position (in contrast agar contains 3,6-anhydro-α-L-galactose). The nongelling λ fraction is composed of 1,3-glycosidically linked D-galactose-2-sulfate and 1,4-bonded D-galactose-2,6-disulfate radicals and is slightly soluble in cold water. The λ-carrageenan constructed from D-galactose-4-sulfate in 1,3-bonding and 3,6-anhydro-α-D-galactose-2-sulfate in 1,4-bonding is both water-soluble and gel-forming. Further carrageen types are likewise designated with Greek letters: α, β, γ, μ, v, ζ, π, ω, χ. The type of cations present (K⁺, NH₄⁺, Na⁺, Mg²⁺, Ca²⁺) also influences the solubility of the carrageens.

The use of chitosan in cosmetic preparations is known per se. The chitosan is a partially deacylated chitin. This biopolymer, inter alia, has film-forming properties and is distinguished by a silky feeling on the skin. A disadvantage, however, is its strong adhesiveness to the skin, which occurs in particular—temporarily—during use. In the isolated case, corresponding preparations may not be marketable, since they are not accepted by the consumer or are adversely assessed. As is known, chitosan is employed, for example, in haircare. It is better suited than the chitin on which it is based as a thickener or stabilizer and improves the adhesion and water resistance of polymeric films. Representative of a large number of places where it is found in the prior art: H. P. Fiedler, “Lexicon der Hilfstoffe für Pharmazie, Kosmetik und angrenzende Gebiete” [Encyclopedia of excipients for pharmacy, cosmetics and related fields], third edition 1989, Editio Cantor, Aulendorf, p. 293, keyword “chitosan”.

Chitosan is characterized by the following structural formula:

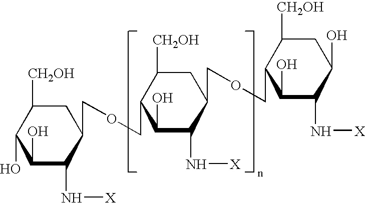

n here assumes values of up to about 10,000, X is either the acetyl radical or hydrogen. Chitosan is formed by deacetylation and partial depolymerization (hydrolysis) of chitin, which is characterized by the structural formula

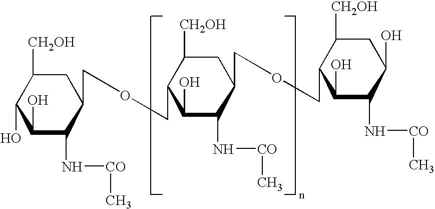

Chitin is an essential constituent of the ectoskeleton ['oχItωv=Greek: armored coat] of the Arthropoda (for example insects, crustaceans, spiders) and is also found in supportive tissues of other organisms (for example molluscs, algae, fungi).

In the range from approximately pH<6, chitosan is positively charged and is also soluble in aqueous systems there. It is not compatible with anionic raw materials. Therefore the use of nonionic emulsifiers suggests itself for the preparation of chitosan-containing oil-in-water emulsions. These are known per se, for example from EP 0 776 657 A1.

According to the invention, chitosans having a degree of deacetylation of >25% are preferred, in particular >55 to 99% [determined by means of ¹H-NMR]).

It is advantageous to choose chitosans having molecular weights between 10,000 and 1,000,000, in particular those having molecular weights between 100,000 and 1,000,000. [determined by means of gel permeation chromatography].

Polyacrylates are likewise gelators to be used advantageously within the meaning of the present invention. Advantageous polyacrylates according to the invention are acrylate/alkyl acrylate copolymers, in particular those which are chosen from the group consisting of the “carbomers” or “carbopols” (Carbopol® is in actual fact a registered mark of the B. F. Goodrich company). In particular, the acrylate/alkyl acrylate copolymer(s) advantageous according to the invention are distinguished by the following structure:

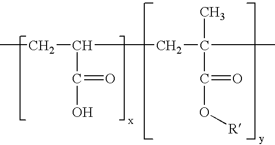

In this structure, R′ is a long-chain alkyl radical and x and y are numbers which symbolize the respective stoichiometric component of the respective comonomer.

Particularly preferred according to the invention are acrylate copolymers and/or acrylate/alkyl acrylate copolymers which are obtainable from the B. F. Goodrich Company under the trade names Carbopol® 1382, Carbopol® 981 and Carbopol® 5984, preferably polyacrylates from the group consisting of the carbopols of the types 980, 981,1382, 2984, 5984 and particularly preferably Carbomer 2001.

Copolymers of C₁₀-C₃₀-alkyl acrylates and one or more monomers of acrylic acid, methacrylic acid or their esters which are crosslinked with an allyl ether of sucrose or an allyl ether of pentaerythritol are furthermore advantageous.

Compounds are advantageous which carry the INCl name “acrylates/C₁₀-C₃₀-alkyl acrylate crosspolymer”. Those obtainable under the trade names Pemulen TR 1 and Pemulen TR 2 from the B. F. Goodrich Company are particularly advantageous.

Compounds are advantageous which carry the INCl name ammonium acryloyldimethyltaurate/vinylpyrrolidone copolymer.

According to the invention, advantageously the ammonium acryloyldimethyltaurate/vinylpyrrolidone copolymers have the empirical formula [C₇H₁₈N₂SO₄]ₙ [C₆H₉NO]ₘ, corresponding to a random structure as follows

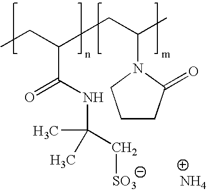

Preferred species within the meaning of the present invention are deposited in Chemical Abstracts under the registry numbers 58374-69-9, 13162-05-5 and 88-12-0 and are obtainable under the trade name Aristoflex® AVC of Clariant GmbH.

Furthermore advantageous are copolymers/crosspolymers comprising acryloyldimethyltaurate, such as, for example, Simugel® EG or Simugel® EG from Seppic S. A.

Further hydrocolloids to be used advantageously according to the invention are also

1. anionic polyurethanes which are soluble or dispersible in water, which are advantageously obtainable from

i) at least one compound which contains two or more active hydrogen atoms per molecule,

ii) at least one diol containing acid or salt groups and

iii) at least one diisocyanate.

The component i) is in particular diols, aminoalcohols, diamines, polyesterols, polyetherols having a number average molecular weight of in each case up to 3000 or their mixtures, where up to 3 mol % of the compounds mentioned can be replaced by triols or triamines. Diols and polyesterdiols are preferred. In particular, the component (a) comprises at least 50% by weight, based on the total weight of the component (a), of a polyesterdiol. Suitable polyester diols are all those which are customarily employed for the preparation of polyurethanes, in particular reaction products of phthalic acid and diethylene glycol, isophthalic acid and 1,4-butanediol, isophthalic acid/adipic acid and 1,6-hexanediol, and adipic acid and ethylene glycol or 5-NaSO₃-isophthalic acid, phthalic acid, adipic acid and 1,6-hexanediol.

Utilizable diols are, for examples, ethylene glycol, propylene glycol, butylene glycol, neopentyl glycol, polyetherols, such as polyethylene glycols having molecular weights up to 3000, block copolymers of ethylene oxide and propylene oxide having number-average molecular weights of up to 3000 or block copolymers of ethylene oxide, propylene oxide and butylene oxide which contain the alkylene oxide units randomly distributed or in the form of blocks. Ethylene glycol, neopentyl glycol, di-, tri-, tetra-, penta- or hexaethylene glycol are preferred. Utilizable diols are moreover poly(a-hydroxycarboxylic acid)diols.

Suitable aminoalcohols are, for example, 2-aminoethanol, 2-(N-methylamino)-ethanol, 3-aminopropanol or 4-aminobutanol.

Suitable diamines are, for example, ethylenediamine, propylenediamine, 1,4-diaminobutane and 1,6-diaminohexane, and α,ω-diamines which can be prepared by amination of polyalkylene oxides with ammonia.

The component ii) is in particular dimethylolpropanoic acid or compounds of the formulae

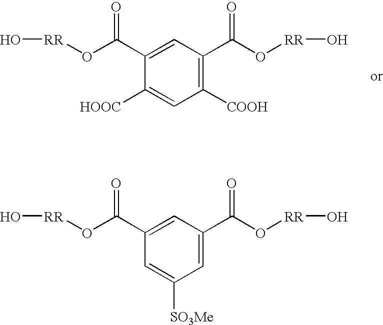

in which RR in each case is a C₂-C₁₈-alkylene group and Me is Na or K.

The component iii) is in particular hexamethylene diisocyanate, isophorone diisocyanate, methyldiphenyl isocyanate (MDI) and/or toluylene diisocyanate.

The polyurethanes are obtainable by reacting the compounds of groups i) and ii) with the compounds of group iii) under an inert gas atmosphere in an inert solvent at temperatures from 70 to 130° C. This reaction can optionally be carried out in the presence of chain extenders in order to prepare polyurethanes having higher molecular weights. As customary in the preparation of polyurethanes, the components [(i)+(ii)]:iii) are advantageously employed in a molar ratio of 0.8 to 1.1:1. The acid number of the polyurethanes is determined from the composition and the concentration of the compounds of the component (ii) in the mixture of the components (i)+(ii).

The polyurethanes have K values according to H. Fikentscher (determined in 0.1% strength by weight solutions in N-methylpyrrolidone at 25° C. and pH 7) of 15 to 100, preferably 25 to 50.

The K value, also designated as intrinsic viscosity, can be determined simply by viscosity measurements of polymer solutions and is therefore a frequently used parameter in the technical field for the characterization of polymers. For a certain type of polymer, it is assumed under standardized measuring conditions to be solely dependent on the mean molar mass of the sample investigated and calculated by means of the relationship K value=1000 k according to the Fikentscher equation.

$$k = \frac{1.51 g {η}_{r} - 1 \pm \sqrt{1 + ( \frac{2}{c} + 2 + 1.51 g {η}_{r} ) \cdot 1.51 g {η}_{r}}}{150 + 300 c}$$

In which:

ηᵣ=relative viscosity (dynamic viscosity of the solution/dynamic viscosity of the solvent) and

c=mass concentration of polymer in the solution (in g/cm³).

The polyurethanes containing acid groups are, after neutralization, (partially or completely) water-soluble or dispersible without the aid of emulsifiers. As a rule, the salts of the polyurethanes have a better water solubility or dispersibility in water than the unneutralized polyurethanes. As a base for the neutralization of the. polyurethanes, it is possible to use alkali metal bases such as sodium hydroxide solution, potassium hydroxide solution, sodium carbonate, sodium hydrogen-carbonate, potassium carbonate or potassium hydrogencarbonate and alkaline earth metal bases such as calcium hydroxide, calcium oxide, magnesium hydroxide or magnesium carbonate and also ammonia and amines. 2-Amino-2-methylpropanol, dimethylolaminopropylamine and triisopropanolamine have proven particularly suitable for the neutralization of the polyurethanes containing acid groups. The neutralization of the polyurethanes containing acid groups can also be carried out with the aid of mixtures of two or more bases, for example mixtures of sodium hydroxide solution and triisopropanolamine. Depending on the intended use, the neutralization can be carried out partially, for example to 20 to 40% or completely, i.e. to 100%.

These polymers and their preparation are described in more detail in DE-A-42 25 045, to which reference is fully made hereby.

2. Cationic polyurethanes and polyureas soluble or dispersible in water, comprising

a) at least one diisocyanate, which can already have been reacted beforehand with one or more compounds which contain two or more active hydrogen atoms per molecule, and

b) at least one diol, primary or secondary aminoalcohol, primary or secondary diamine or primary or secondary triamine having one or more tertiary, quaternary or protonated tertiary amino nitrogen atoms.

Preferred diisocyanates are indicated as above under 1). Compounds having two or more active hydrogen atoms are diols, aminoalcohols, diamines, polyesterols, polyamidediamines and polyetherols. Suitable compounds of this type are as indicated above under 1).

The polyurethanes are prepared as described above under 1). Charged cationic groups can be produced in the polyureas from the tertiary amino nitrogen atoms present either by protonation, for example using carboxylic acids we lactic acid, or by quaternization, for example using alkylating agents such as C₁- to C₄-alkyl halides or sulfates. Examples of such alkylating agents are ethyl chloride, ethyl bromide, methyl chloride, methyl bromide, dimethyl sulfate and diethyl sulfate.

These polymers and their preparation are described in more detail in DE 42 41 118 Al, to which reference is fully made hereby.

3. Linear polyurethanes having carboxylate groups from

i) a 2,2-hydroxymethyl-substituted carboxylic acid of the formula

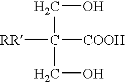

in which RR′ is a hydrogen atom or a C₁-C₂₀-alkyl group, which is used in an amount which suffices that in the polyurethane 0.35 to 2.25 milliequivalents of carboxyl groups are present per g of polyurethane,

ii) 10 to 90% by weight, based on the weight of the polyurethane, of one or more organic compounds having not more than two active hydrogen atoms and

iii) one or more organic diisocyanates.

The carboxyl groups contained in the polyurethane are finally at least partially neutralized using a suitable base. These polymers and their preparation are described in EP 0 619 111 A1, to which reference is fully made hereby.

4. Carboxyl-containing polycondensation products of anhydrides of tri- or tetra-carboxylic acids and diols, diamines or aminoalcohols (polyesters, polyamides or polyester amides). These polymers and their preparation are described in more detail in DE 42 24 761 A1, to which reference is fully made hereby.

5. Polyacrylates and polymethacrylates, such as are described in more detail in DE 43 14 305 A1, DE 36 27 970 A1 and DE 29 17 504 A1. Reference to these publications is fully made hereby.

The polymers used according to the invention preferably have a K value of 25 to 100, preferably 25 to 50. The polymers are contained in the composition according to the invention in general in an amount in the range from 0.2 to 20% by weight, based on the total weight of the composition. The salt is used in an amount effective for improving the exchangeability of the polymers. In general, the salt is employed in an amount from 0.02 to 10% by weight, preferably 0.05 to 5% by weight and in particular 0.1 to 3% by weight, based on the total weight of the composition.

The total amount of one or more hydrocolloids in the finished cosmetic or dermatological preparations is advantageously chosen to be less than 5% by weight, preferably between 0.1 and 1.0% by weight, based on the total weight of the preparations.

It has furthermore turned out to be very preferred if up to 50% by weight of silicone resins are added to the matrix, in particular between 5 and 40% by weight.

The silicone resins usually used in industry are polymethyl- or polymethylphenyl-siloxanes which are crosslinked to a greater or lesser extent, and whose elasticity and resistance to heat increases with the content of phenyl groups. Pure methylsilicone resins are relatively brittle and moderately heat-resistant. The long-term resistance to heat is high (180 to 200° C.). The silicone resins usually come onto the market in precondensed form.

A matrix according to the invention when used as a wound dressing combines the positive silicone properties such as adhesive behavior and skin compatibility with the advantage of an absorption capacity for wound exudation (water), which is adjustable over a wide range. Additionally, the matrix can be doped with active compounds which can be delivered from the matrix to the wound via the wound exudation channels formed.

One possible area of application is in the care of 3rd degree burns. In the care of such burns three problems occur:

1. Owing to the destroyed skin layer, relatively large amounts of moisture can escape unhindered.

2. The open tissue layers are an ideal breeding ground for germs.

3. Burn wounds are prone to severe scar formation on healing.

A matrix according to the invention reduces these problems simultaneously:

1. The water absorption capacity can be adjusted according to the natural release of moisture of the skin.

2. By incorporation of a disinfectant into the matrix such as, for example, 1% by weight of chlorhexidine diacetate, an infection of the wound can be counteracted.

3. Silicone wound dressings reduce the scar formation.

As an additional positive effect, on changing the wound dressing necrotic tissue is gently removed by adhesion to the wound dressing.

Aside from for wound treatment as described above, the hydroactive silicone matrix can also be employed as a topical medicament (TTS) or as a cosmetic for skin treatment. In this connection, the active compounds are incorporated directly into the matrix, or in the case of possible interactions between active compound and silicone crosslinkage subsequently introduced into the matrix as a solution.

Transdermal therapeutic systems which are doped with ethereal oils and their constituents (for example eucalyptus oil, peppermint oil, camphor, menthol), possess a long-term, therapeutic effect in the case of common colds, headaches and further indications.

Among ethereal oils, concentrates obtained from plants are known which are employed as natural raw materials mainly in the perfume and foodstuffs industries and which consist to a greater or lesser extent of volatile compounds, such as, for example, true ethereal oils, citrus oils, essential oils, oleoresins.

Often, the term is also used for the volatile constituents still contained in the plants. In the true sense, however, ethereal oils are understood as meaning mixtures of volatile components which are prepared from plant raw materials by steam distillation.

True ethereal oils consist exclusively of volatile components whose boiling point are mainly between 150 and 300° C. Unlike, for example, fatty oils, they therefore do not leave behind any lasting transparent fatty spot on dabbing onto filter paper. Ethereal oils mainly contain hydrocarbons or monofunctional compounds such as aldehydes, alcohols, esters, ethers and ketones.

Stock compounds are mono- and sesquiterpenes, phenylpropane derivatives and relatively long-chain aliphatic compounds.

In some ethereal oils one constituent dominates (for example eugenol in oil of cloves with more than 85%), others are again of extremely complex composition. Often, the organoleptic properties are not determined by the main constituents, but by secondary or trace constituents, such as, for example, by the 1,3,5-undecatrienes and pyrazines in galbanum oil. In many of the commercially important ethereal oils, the number of identified components runs into hundreds. Very many constituents are chiral, very often one enantiomer predominating or being exclusively present, such as, for example, (−)-menthol in peppermint oil or (−)-linalyl acetate in lavender oil.

In one advantageous embodiment, the matrix contains 1 to 10% by weight of ethereal oils, which are chosen in particular from the group consisting of eucalyptus oil, peppermint oil, camomile oil, camphor, menthol, citrus oil, oil of cinnamon, oil of thyme, lavender oil, oil of cloves, tea tree oil, cajuput oil, niaouli oil, kanuka oil, manuka oil, templin oil.

Citrus oils are ethereal oils which are obtained from the peel of citrus fruits (bergamot, grapefruit, lime, tangerine, orange, lemon), often also called agrumen oils.

Citrus oils consist to a large part of monoterpene hydrocarbons, mainly limonene (exception: bergamot oil, which contains only about 40%).

Camphor is understood as meaning 2-bornanone, 1,7,7-trimethylbicyclo[2.2.1]-heptan-2-one, see figure below.

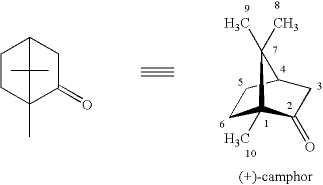

Peppermint oils are ethereal oils obtained by steam distillation from leaves and inflorescences of various peppermint varieties, occasionally also those from Mentha arvensis.

Menthol has three asymmetric C atoms and accordingly occurs in four diastereomeric pairs of enantiomers (cf. the formula images, the other four enantiomers are the corresponding mirror images).

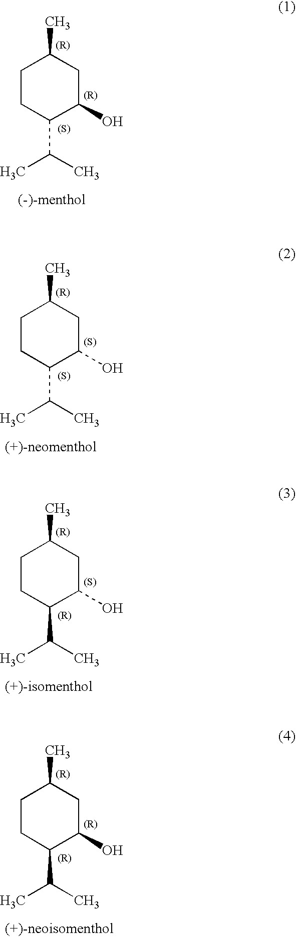

The diastereomers, which can be separated by distillation, are designated as neoisomenthol, isomenthol, neomenthol [(+)-form: constituent of Japanese peppermint oil] and menthol. The most important isomer is (−)-menthol (levomenthol), lustrous prisms having a strongly peppermint-like smell.

Menthol produces a pleasant sensation of coolness in migraine and the like on rubbing into the skin (particularly on the forehead and temples) as a result of surface anesthesia and stimulation of the cold-sensitive nerves; actually, the sites concerned show a normal or elevated temperature. The other isomers of menthol do not possess these effects.

In a further advantageous embodiment of the silicone-based moisture-absorbing matrix, superabsorbers are added to this.

The matrix then contains in a further advantageous embodiment a particularly hydrophilic filler based on cellulose and its derivatives, whose mean particle size is in the range from 20 to 60 μm, because in the selection of the fillers it has surprisingly been found that silica- or cellulose-based fillers are particularly suitable, the latter possessing an isotropic shape and tending not to swell on contact with water. In this context, fillers having a particle size of less than or equal to 100 μm are particularly suitable.

The use of hydrophilic fillers in a nonpolar matrix is known in the literature. They are described explicitly for use in transdermal therapeutic systems in EP 0 186 019 A1. Here, however, only up to a concentration of 3 to 30% by weight, without details regarding these fillers being mentioned. Experience shows that systems having a filler content of over 30% by weight markedly lose adhesiveness and become hard and brittle. As a result, they lose the basic requirement of a transdermal therapeutic system.

Preferably, microcrystalline or amorphous cellulose-based fillers are employed in significantly higher concentrations without an adverse influencing of the adhesive technological properties occurring, in particular if they possess an isotropic shape having a particle size of not greater than 100 μm. Higher contents of fillers are desirable for improving the wearing properties, in particular in the case of long-lasting and repeated application.

In addition, permeation-promoting constituents in the concentration range up to 30% by weight, preferably 5 to 15% by weight, are preferably added to the silicone-based moisture-absorbing matrix.

These include, for example, lipophilic solubilizers/enhancers such as decyl oleate, isopropyl myristate and isopropyl palmitate (IPM and IPP), 2-octyldodecanol etc.

In addition, skin-caring, cosmetic additives can advantageously be added to the silicone-based moisture-absorbing matrix, particularly to 0.2 to 10% by weight, very particularly to 0.5 to 5% by weight.

According to the invention, the skin-caring, cosmetic additives (one or more compounds) can very advantageously be chosen from the group consisting of the lipophilic additives, in particular from the following group:

acetylsalicylic acid, atropine, azulene, hydrocortisone and its derivatives, for example hydrocortisone-17-valerate, vitamins, for example ascorbic acid and its derivatives, vitamins of the B and D series, very favorably vitamin B₁, vitamin B₁₂, vitamin D₁, but also bisabolol, unsaturated fatty acids, especially the essential fatty acids (often also called vitamin F), in particular gamma-linolenic acid, oleic acid, eicosapentaenoic acid, docosahexaenoic acid and their derivatives, chloramphenicol, caffeine, prostaglandins, thymol, camphor, extracts or other products of vegetable and animal origin, for example evening primrose oil, borage oil or carob bean oil, fish oils, cod-liver oil but also ceramides and ceramide-like compounds et cetera.

It is also advantageous to choose the additives from the group consisting of the refatting substances, for example purcellin oil, Eucerit® and Neocerit®.

The additive(s) further chosen from the group consisting of the NO synthase inhibitors are particularly advantageous, in particular if the preparations according to the invention are to be used for the treatment and prophylaxis of the symptoms of intrinsic and/or extrinsic skin aging and for the treatment and prophylaxis of the harmful effects of ultraviolet radiation on the skin.

A preferred NO synthase inhibitor is nitroarginine.

The additive(s) chosen from the group which comprises catechols and bile acid esters of catechols and aqueous or organic extracts of plants or parts of plants which contain catechols or bile acid esters of catechols are furthermore advantageous, such as, for example, the leaves of the plant family Theaceae, in particular the species Camellia sinensis (green tea). Their typical constituents are particularly advantageous (such as, for example, polyphenols or catechols, caffeine, vitamins, sugars, minerals, amino acids, lipids).

Catechols are a group of compounds which are to be interpreted as hydrogenated flavones or anthocyanidins and derivatives of “catechol” (3,3′,4′,5,7-flavanpentaol, 2-(3,4-dihydroxyphenyl)chroman-3,5,7-triol). Epicatechol ((2R,3R)-3,3′,4′, 5,7-flavanpentaol) is also an advantageous additive within the meaning of the present invention.

Plant extracts containing catechols are furthermore advantageous, in particular extracts of green tea, such as, for example, extracts of leaves of the plants of the species Camellia spec., very particularly the tea strains Camellia sinenis, C. assamica, C. taliensis or C. irrawadiensis and crossings of these with, for example, Camellia japonica.

Preferred additives are further polyphenols or catechols from the group consisting of (−)-catechol, (+)-catechol, (−)-catechol gallate, (−)-gallocatechol gallate, (+)-epicatechol, (−)-epicatechol, (−)-epicatechol gallate, (−)-epigallocatechol, (−)-epigallocatechol gallate.

Flavone and its derivatives (often also collectively called “flavones”) are also advantageous additives within the meaning of the present invention. They are characterized by the following basic structure (substitution positions indicated):

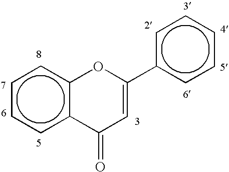

Some of the more important flavones, which can also preferably be employed in the preparations according to the invention, are listed in the table below:

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kampferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones as a rule occur in glycosidated form.

According to the invention, the flavonoids are preferably chosen from the group consisting of the substances of the generic structural formula

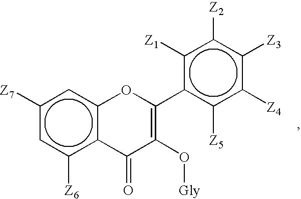

where Z₁ to Z₇ independently of one another are chosen from the group consisting of H, OH, alkoxy and hydroxyalkoxy groups, where the alkoxy or hydroxyalkoxy groups can be branched or unbranched and can have 1 to 18 C atoms, and where Gly is chosen from the group consisting of the mono- and oligoglycoside radicals.

According to the invention, the flavonoids, however, can also advantageously be chosen from the group consisting of the substances of the generic structural formula

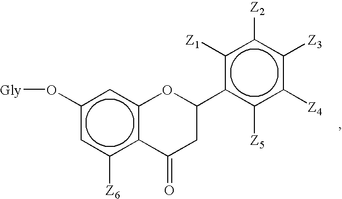

where Z₁ to Z₆ independently of one another are chosen from the group consisting of H, OH, alkoxy and hydroxyalkoxy groups, where the alkoxy or hydroxyalkoxy groups can be branched or unbranched and can have 1 to 18 C atoms, and where Gly is chosen from the group consisting of the mono- and oligoglycoside radicals.

Preferably, those structures can be chosen from the group consisting of the substances of the generic structural formula

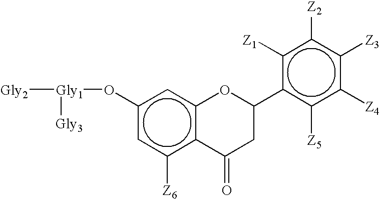

where Gly₁, Gly₂ and Gly₃ independently of one another are monoglycoside radicals. Gly₂ and Gly₃ can also individually or together be saturations by hydrogen atoms.

Preferably, Gly₁, Gly₂ and Gly₃ independently of one another are chosen from the group consisting of the hexosyl radicals, in particular the rhamnosyl radicals and glucosyl radicals. However, other hexosyl radicals, for example allosyl, altrosyl, galactosyl, gulosyl, idosyl, mannosyl and talosyl are optionally also to be used advantageously. It can also be advantageous according to the invention to use pentosyl radicals.

Advantageously, Z₁ to Z₅ independently of one another are chosen from the group consisting of H, OH, methoxy, ethoxy and 2-hydroxyethoxy groups, and the flavone glycosides have the structure

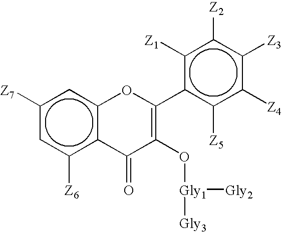

Particularly advantageously, the flavone glycosides according to the invention are from the group which are represented by the following structure:

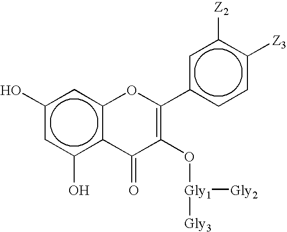

where Gly₁, Gly₂ and Gly₃ independently of one another are monoglycoside radicals. Gly₂ and Gly₃ can also individually or together be saturations by hydrogen atoms.

Preferably, Gly₁, Gly₂ and Gly₃ independently of one another are chosen from the group consisting of the hexosyl radicals, in particular the rhamnosyl radicals and glucosyl radicals. However, other hexosyl radicals, for example allosyl, altrosyl, galactosyl, gulosyl, idosyl, mannosyl and talosyl are optionally also to be used advantageously. It can also be advantageous according to the invention to use pentosyl radicals.

It is particularly advantageous within the meaning of the present invention to choose the flavone glycoside(s) from the group consisting of α-glucosylrutin, α-glucosylmyricetin, α-glucosylisoquercitrin, α-glucosylisoquercetin and α-glucosylquercitrin.

According to the invention, α-glucosylrutin is particularly preferred.

According to the invention, also advantageous are naringin (aurantiin, naringenin-7-rhamnoglucoside), hesperidin (3′,5,7-trihydroxy-4′-methoxyflavanone-7-rutinoside, hesperidoside, hesperetin-7-O-rutinoside). Rutin (3,3′,4′,5,7-pentahydroxyflyvone-3-rutinoside, quercetin-3-rutinoside, sophorin, birutan, rutabion, taurutin, phytomelin, melin), troxerutin (3,5-dihydroxy-3′,4′, 7-tris(2-hydroxyethoxy)flavone-3-(6-O-(6-deoxy-α-L-mannopyranosyl)-β-D-glucopyranoside)), monoxerutin (3,3′,4′,5-tetrahydroxy-7-(2-hydroxyethoxy)flavone-3-(6-O-(6-deoxy-α-L-mannopyranosyl)-β-D-glucopyranoside)), dihydrorobinetin (3,3′,4′,5′,7-pentahydroxyflavanone), taxifolin (3,3′,4′,5,7-penta-hydroxyflavanone), eriodictyol-7-glucoside (3′,4′,5,7-tetrahydroxyflavanone-7-glucoside), flavanomarein (3′,4′,7,8-tetrahydroxyflavanone-7-glucoside) and isoquercetin (3,3′,4′,5,7-pentahydroxyflavanone-3-(β-D-glucopyranoside) are also advantageous.

It is also advantageous to choose the additive(s) from the group consisting of the ubiquinones and plastoquinones.

Ubiquinones are distinguished by the structural formula

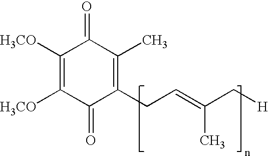

and are the most widespread and thus the best investigated bioquinones. Depending on the number of the isoprene units linked in the side chain, ubiquinones are designated as Q-1, Q-2, Q-3 etc or on the number of C atoms as U-5, U-10, U-15 etc. They preferably occur with certain chain lengths, for example in some microorganisms and yeasts with n=6. In most mammals including man Q10 predominates.

Particularly advantageous is coenzyme Q10, which is characterized by the following structural formula:

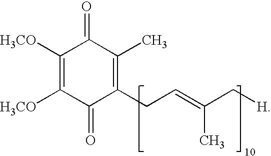

Plastoquinones have the general structural formula

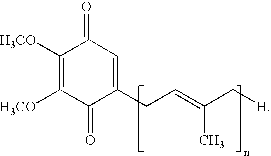

Plastoquinones differ in the number n of the isoprene radicals and are designated accordingly, for example PQ-9 (n=9). Furthermore, other plastoquinones having different substituents on the quinone ring exist.

Creatine and/or creatine derivatives are also preferred additives within the meaning of the present invention. Creatine is distinguished by the following structure:

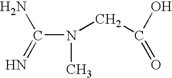

Preferred derivatives are creatine phosphate and creatine sulfate, creatine acetate, creatine ascorbate and the derivatives esterified on the carboxyl group by mono- or polyfunctional alcohols.

A further advantageous additive is L-carnitine [3-hydroxy4-(trimethylammonio)butyric acid betaine]. Acylcarnitines, which chosen from the group of substances of the following general structural formula

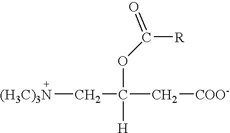

where R is chosen from the group consisting of the branched and unbranched alkyl radicals having up to 10 carbon atoms are also advantageous additives within the meaning of the present invention. Propionylcarnitine and in particular acetylcarnitine are preferred. Both enantiomers (D- and L-form) can be used advantageously within the meaning of the present invention. It can also be advantageous to use any desired mixtures of enantiomers, for example a racemate of the D- and L-form.

Further advantageous additives are sericoside, pyridoxol, vitamin K, biotin and aromatic substances.

The list of the additives or additive combinations mentioned, which can be used in the preparations according to the invention, should of course not be limiting. The additives can be used individually or in any desired combinations with one another.

Pharmaceutically active substances can then be added to the matrix of the active compound-containing matrix patch, preferably up to 40% by weight, particularly to 0.1 to 25% by weight, very particularly to 0.5 to 10% by weight.

Typical active compounds are—without making the claim to completeness in the context of the present invention:

| Indication: | Active compound |
|---|---|
| Antimycotics | Nafitin |
| | Amorrolfin |
| | Tolnaftate |
| | Ciclopirox |
| Antiseptics | Thymol |
| | Eugenol |
| | Triclosan |
| | Hexachlorophene |
| | Benzalkonium chloride |
| | Clioquinol |
| | Quinolinol |
| | Undecenoic acid |
| | Ethacridine |
| | Chlorhexidine |
| | Hexetidine |
| | Dodicine |
| | Iodine |
| Nonsteroidal | Glycol salicylate |
| antirheumatics | Flufenamic acid |
| | Ibuprofen |
| | Etofenamate |
| | Ketoprofen |
| | Piroxicam |
| | Indomethacin |
| Antipuriginous agents | Polidocanol |
| | Isoprenaline |
| | Crotamiton |
| Local anesthetics | Benzocaine |
| Antipsoriatics | Ammonium bitumasulfonate |
| Keratolytics | Urea |
| | Salicylic acid |

In addition, hyperemizing active compounds such as natural active compounds of cayenne pepper or synthetic active compounds such as nonivamide, nicotinic acid derivatives, preferably benzyl nicotinate or propyl nicotinate, can also be mentioned or antiinflammatories and/or analgesics.

By way of example, capsaicin

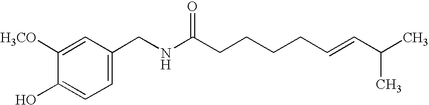

[8-methyl-trans-6-nonenoic acid (4-hydroxy-3-methoxybenzylamide)]

Nonivamide

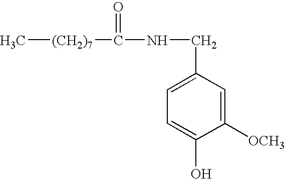

Benzyl nicotinate

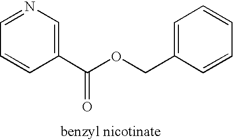

may be mentioned.

Of particular importance among the active compounds, the disinfectants and antiseptics are to be emphasized, such that their use in the matrix should again be stressed.

Disinfectants are designated as substances which are suitable for the disinfection, i.e. for the control, of pathogenic microorganisms (for example bacteria, viruses, spores, micromycetes and mold fungi), to be precise in general by application to the surface of skin, clothing, equipment, rooms, but also of drinking water, foodstuffs, seed (dressings) and as soil disinfectants.

Disinfectants to be applied particularly locally, for example for wound disinfection, are also designated as antiseptics.

Disinfectants are defined as substances or substance mixtures which when used on articles or surfaces convert these into a state in which they no longer cause infection. Their action must be bactericidal, fungicidal, virucidal and sporicidal (collective term: microbicidal). An effect in the sense of bacteriostasis is inadequate for disinfectants. They are therefore in general pantoxic, i.e. they display their action against all living cells.

Depending on the intended use, the disinfectants are divided into those for washing, surface, instruments, skin and hands, and for stool and sputum disinfection. Disinfectant cleansers are understood as meaning those disinfectants which also function as cleansers and, if appropriate, toiletry preparations.

Taking into consideration the various demands which are made on disinfectants, such as, for example, broad spectrum of action, short times of action, skin compatibility, low toxicity, material compatibility etc, only a few types of active compound are suitable for use:

1. The aldehydes (formaldehyde, glyoxal, glutaraldehyde) are the most important active compound group. They possess a broad spectrum of action including virus activity and sporicidal action in the case of formaldehyde and glutaraldehyde.

2. Phenol derivatives possess a good bactericidal action, but are not sporicidal. Compared with almost all other disinfectant active compounds, they have the advantage of being comparatively only slightly influenced by dirt. They are therefore better suited for stool disinfection. Typical representatives are 2-biphenylol and p-chloro-m-cresol (4-chloro-3-methylphenol).

3. Alcohols are distinguished by rapid activity, but only at relatively high concentrations of about 40-80%.

4. The quaternary ammonium compounds, cationic surfactants (invert soaps) and amphosurfactants belong to the class consisting of the surfactants. They are distinguished by fairly good skin and material compatibility and odor neutrality. Their spectrum of action, however, is only limited. Benzalkonium chloride, cetrimonium bromide, cetylpyridinium chloride (hexadecylpyridinium chloride) and others, for example, are included here.

Quaternary ammonium compounds are organic ammonium compounds having quaternary nitrogen atoms. Quaternary ammonium compounds having a hydrophobic alkyl radical are biocidal; their use is certainly declining for toxicological reasons.

Quaternary ammonium compounds are prepared by reaction of tertiary amines with alkylating agents, such as, for example, methyl chloride, benzyl chloride, dimethyl sulfate, dodecyl bromide, but also ethylene oxide. Depending on the tert-amine employed, three groups are distinguished:

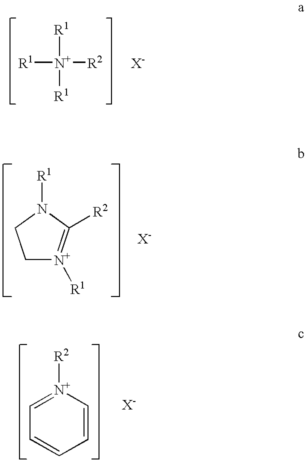

a) Linear alkylammonium compounds

b) Imidazolinium compounds

c) Pyridinium cpds. R¹═CH₃, R²═C₈₋₁₈, X=halogen.

The alkylation of tertiary amines having a long alkyl radical and two methyl groups takes place particularly easily, the quaternization of tertiary amines having two long radicals and a methyl group can also be carried out under mild conditions with the aid of methyl chloride. Amines which have three long alkyl radicals or hydroxy-substituted alkyl radicals are not very reactive and are preferably quaternized using dimethyl sulfate.

5. Of the halogens, chlorine and iodine have a certain importance as disinfectants. Chlorine is known from water treatment and swimming pool disinfection and therewith its unpleasant properties such as odor and corrosiveness. In spite of the excellent action against bacteria, fungi, spores and viruses, chlorine-containing disinfectants have not found any widespread use in the human field for the above-mentioned reasons and because of the heavy chlorine loss due to organic substances. In contrast, hypochlorites, hypochlorous and chloroisocyanuric acids are still extensively used as industrial disinfectants. Tincture of iodine is used in the medical field as an antiseptic.

6. Disinfectants based on active oxygen (for example hydrogen peroxide, peroxyacetic acid) have recently again gained some importance.

Aside from the microbicidal active compounds mentioned, a number of microbistatic substances and preservatives (diphenyl ether, carbanilides, acetanilides of aromatic acids and their salts) for specific use are still on the market, which are included in the broader sense with the disinfectants.

A uniform mode of action of the disinfectants cannot be discerned. Whereas some preparations should act destructively on the cytoplasmic membrane of the bacteria, an irreversible blocking of important sulfide bonds in enzymes or of trace elements (by chelation) is assumed by others.

The invention accordingly also relates to the use of disinfectant systems which contain

at least one nonionic surfactant and

at least one amino acid and/or an amino acid derivative

and at least one disinfectant agent and/or a microbicidal active compound.

Advantageously, the nonionic surfactant(s) are chosen from the group consisting of the alkyl ethoxylates and/or alkyl propxylates, whose alkyl group is a saturated or unsaturated, straight- or branched-chain alkyl group having (8) 10 to 18, preferably 12 to 14 carbon atoms, where they preferably contain, per molecule, 2 to 15, in particular 5 to 9, especially 7, ethylene oxide units. Isotridecanol ethoxylate and/or fatty alcohol polyglycol ethers are very particularly preferred.

Advantageously, the total amount of nonionic surfactants (one or more compounds) is chosen from the range from 1.0 to 20.0% by weight, preferably from 5.0 to 15.0% by weight, in each case based on the total weight of the matrix.

Advantageous amino acids are, for example, glutamic acid, which is distinguished by the following structural formula:

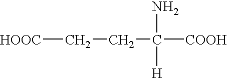

and/or pyrrolidonecarboxylic acid (pyroglutamic acid), which is distinguished by the following structural formula:

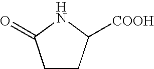

Advantageously, the total amount of amino acids (one or more compounds) is chosen from the range from 0.1 to 10.0% by weight, preferably from 0.5 to 2.0% by weight, in each case based on the total weight of the matrix.

The disinfectant agent(s) (microbicidal active compounds) are preferably chosen from the group consisting of the aldehydes (for example formaldehyde, glyoxal, glutaraldehyde), the phenol derivatives (for example 2-biphenylol and p-chloro-m-cresol (4-chloro-3-methylphenol), the alcohols, the quaternary ammonium compounds (for example benzalkonium chloride, cetrimonium bromide, cetylpyridinium chloride (hexadecylpyridinium chloride). Aldehydes and quaternary ammonium compounds are very particularly preferred here.

In a particularly advantageous embodiment, the disinfectant systems can further contain amphosurfactants. Amphosurfactants are surfactants which possess both acidic and basic hydrophilic groups and thus behave, according to the conditions, acidically or basically. Advantageous amphosurfactants are, for example, those based on aliphatic polyamines having carboxyl, sulfo or phosphono side chains, such as, for example, R—NH—(CH₂)ₙ—COOH.

Preferred amphosurfactants are those, for example, whose alkyl group is a saturated or unsaturated, straight- or branched-chain alkyl group having 10 to 18, preferably 12 to 14, carbon atoms.

Furthermore, amphosurfactants from the group consisting of the amphopropionates are particularly advantageous, such as, for example, cocobetaineamido amphopropionate, which is distinguished by the following structure:

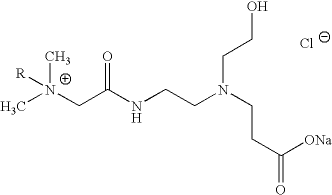

Advantageously, the total amount of amphosurfactants (one or more compounds) is chosen from the range from 1.0 to 10.0% by weight, preferably from 2.0 to 5.0% by weight, in each case based on the total weight of the matrix.

It is advantageous to carry out the dilution such that the content of the individual substances in the use solution is as follows:

| | |
|---|---|
| nonionic surfactants: | between 0.005 and 1% by weight |
| amino acid: | between 0.0005 and 0.5% by weight |
| optionally amphosurfactants: | between 0.005 and 0.5% by weight |
| disinfectant agents: | between 0.1 and 2.0% by weight |

Additionally to the abovementioned components, the ? and disinfectant systems can contain customary preservatives, colorants, fragrances and/or other customary excipients for preparations of this type. However, it is also possible to use those components which display a (preserving, caring etc.) action and in the course of this at the same time provide for a certain color and/or a pleasant fragrance.

The amounts of vehicles and perfume to be employed in each case can easily be determined by the person skilled in the art by simple testing depending on the nature of the particular product.

Also advantageous is the use of disinfectant systems which contain

at least one microbicidal active compound, chosen from the group consisting of the alkylamines

at least one amino acid and/or one amino acid derivative

at least one quaternary ammonium compound.

Advantageously, the quaternary ammonium compounds are preferably chosen from the group consisting of benzalkonium chloride, didecylmethylammonium chloride, cetrimonium bromide, cetylpyridinium chloride (hexadecylpyridinium chloride).

Advantageously, the alkylamine is dodecylbispropylenetriamine.

Advantageous amino acids are, for example, glutamic acid, which is distinguished by the following structural formula:

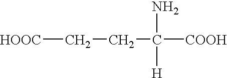

and/or pyrrolidonecarboxylic acid (pyroglutamic acid), which is distinguished by the following structural formula:

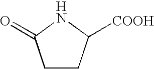

Advantageously, the total amount of amino acids (one or more compounds) is chosen from the range from 0.1 to 10.0% by weight, preferably from 0.5 to 2.0% by weight, in each case based on the total weight of the matrix.

According to the invention, nonionic surfactants are advantageously additionally added, in particular advantageously chosen from the group consisting of the alkyl ethoxylates, whose alkyl group is a saturated or unsaturated, straight- or branched-chain alkyl group having 8 to 18, preferably 12 to 14, carbon atoms, where they preferably contain, per molecule, 2 to 15, in particular 5 to 9, especially 7, ethylene oxide units. Isotridecanol ethoxylate and/or fatty alcohol polyglycol ethers are particularly preferred.

Advantageously, the total amount of nonionic surfactants (one or more compounds) is chosen from the range from 1.0 to 20.0% by weight, preferably from 5.0 to 15.0% by weight, in each case based on the total weight of the matrix.

Furthermore, as agents for disinfection, preservation and antisepsis, a large number of microbicidally active chemical substances or mixtures of these substances are known per se. Microbicidal substances are in general active to a greater or lesser extent against the customary spectrum of microorganisms, such as, for example, gram-positive bacteria, gram-negative bacteria, mycobacteria, yeasts, fungi, viruses and the like, such that an adequate disinfection, preservation or antisepsis can customarily be achieved by suitable active compound combinations.

For disinfection, preservation and antisepsis, a number of active compounds are known, in particular aldehydes, such as, for example, formaldehyde or glutaraldehyde, quaternary ammonium compounds and long-chain amines, phenols or alcohols.

Aldehydes fix residues of blood and protein by chemical reaction to the articles to be disinfected, such that these are difficult to clean after disinfection. Moreover, they have a comparatively high allergenic potential, such that applications to skin and hands are only possible in low concentrations or else suitable in combination with further active compounds in order to be able to keep below the sensitization threshold. Higher concentrations of aldehydes are also undesired because of their odor, such that for this reason too the concentration is lowered by combination with further active compounds.

Quaternary ammonium compounds and long-chain amines are frequently used in bottle disinfection and for manual instrument disinfection and to a small extent also in antisepsis of the hands. In comparison to the aldehydes, the odor of these compounds is markedly less unpleasant. A chemical reaction with proteins does not take place, but physical precipitation of proteins occurs, which can be partially compensated by skillful combination with surfactants. The quaternary ammonium compounds are not suitable for mechanical instrument disinfection, because as a result of the turbulence in the cleaning machine a heavy, undesired formation of foam occurs. In the disinfection of bottles, quaternary ammonium compounds show a strong tendency to form layers of these compounds on the surfaces. A further crucial disadvantage is the restricted spectrum of action of quaternary ammonium compounds, since these act neither sporocidally nor against uncoated viruses.

Phenols are on the decline in nearly all application areas for disinfectants especially because of their odor, their low activity against the poliovirus, their in some cases poor degradability, their high lipid solubility associated with a strong penetration through the skin and toxic and mutagenic risks.

The aliphatic alcohols ethanol, 1-propanol and 2-propanol have long been known as active compounds for the disinfection of skin and hands or for antisepsis of the skin and hands. Using disinfectants and antiseptics based on alcohols, in the case of short times of action of 30 to 60 seconds microorganism count reductions of up to 99.9% can be achieved. A general, abridged presentation of the microbicidal activity of alcohols is found in the book: K. H. Wallhäuβer, “Praxis der Sterilisation, Desinfektion und Konservierung” [Sterilisation, disinfection and preservation practice], G. Thieme Verlag, Stuttgart, N.Y., 5th edition, pp. 469-474.

Alcohols possess a bactericidal action which increases from methanol to propanol. Ethanol, n-propanol and isopropanol are especially used, the alcohol content of the preparations in general being between 50 and 80%. The significant advantage of alcohols is that the onset of action takes place very rapidly. It is disadvantageous that they are not active against spores and that the action ends after a very short time, since alcohols rapidly evaporate. An antiviral activity of alcohols is in fact discussed, but only on the other side of a high concentration limit, which in the case of ethanol is presumed to be at about 80%.

It has been shown in practice that alcoholic disinfectants and antiseptics are not able or not able to an adequate extent to destroy viruses and traces of Bacillus and Clostridium species. Although the freedom from spores of alcoholic solutions can be achieved by filtration, in practice it cannot be completely ruled out that microorganism spores (subsequently) reach the preparations, for example during the short-term opening of the storage vessels or during the filling of the compositions into containers which already contain spores. For this reason, when using alcoholic skin and hand antiseptics there is always a certain risk of an infection caused by spores.

In a particularly advantageous embodiment, the antiseptic is composed as follows:

| | | |
|---|---|---|
| (a) | 42-47% by weight | of 1-propanol |
| (b) | 22-27% by weight | of 2-propanol |
| (c) | 4-6% by weight | of ethanol |
| (d) | at least 20% by weight | of water |
| (e) | at most 0.0001% by weight | of substances which are present as |
| | | solids under normal conditions |
| (f) | no active content | of further substances which are |
| | | distinguished by virucidal properties |

Antiseptics are particularly suitable for the treatment of the skin. Antiseptics show a very good activity against dermatophytes and are surprisingly distinguished in particular in that they have a good activity against viruses.

The constituents of the antiseptics age with respect to their antimicrobial and antiviral properties synergistically, that is in a significant manner superadditively.

Accordingly, also advantageous is the use of a preparation of

| | | |
|---|---|---|
| (a) | 42-47% by weight | of 1-propanol |
| (b) | 22-27% by weight | of 2-propanol |
| (c) | 4-6% by weight | of ethanol |
| (d) | At least 20% by weight | of water |
| (e) | At most 0.0001% by weight | of substances which are present as |
| | | solids under normal conditions |
| (f) | No active content | of further substances which are |
| | | distinguished by virucidal properties |

as an antiseptic, in particular the use for the control or inactivation of the HIV virus or of the hepatitis B virus.

Particularly suitable as an antiseptic, in particular for the oral and pharyngeal cavity, is in turn chlorhexidine,

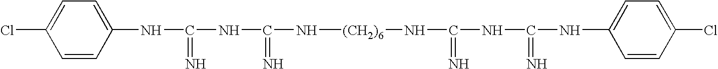

the international nonproprietary name for 1,1-hexamethylene-bis[5-(4-chlorophenyl)biguanide], the dihydrochloride, diacetate and digluconate being used as an antiseptic.

Particularly advantageously, the silicone-based moisture-absorbing matrix is applied to a flexible covering layer, in particular when used as a plaster.

An appropriate plaster is constructed from a carrier such as films, nonwovens, wovens, foams etc, the carrier-anchored silicone matrix and a covering film for the protection of the adhesive matrix before use.

In a further preferred embodiment of the invention, polymer films, nonwovens, wovens fabric and their combinations are employed as carriers. As carrier materials, a choice can be made from, inter alia, polymers such as polyethylene, polypropylene and polyurethane or alternatively natural fibers.

For example, a metallocene-polyethylene nonwoven is suitable.

The metallocene-polyethylene nonwoven preferably has the following properties:

a basis weight of 40 to 200 g/m², in particular of 60 to 120 g/m², and/or

a thickness of 0.1 to 0.6 mm, in particular of 0.2 to 0.5, and/or

a maximum tensile force extension longitudinally of 400 to 700% and/or

a maximum tensile force extension transversely of 250 to 550%.

It is then possible to use as carrier materials known nonwovens which are mechanically consolidated, namely by oversewing with separate threads or by interlooping.

In the first case, the web-yarn stitchbonds result. For the production of these, a nonwoven is introduced which, for example can be diagonally paneled and is oversewn by means of separate threads in pillarstitch or tricot formations.

These nonwovens are known under the name “Maliwatt” (from Malimo) or Arachne. In the second type of consolidation, a cross-laid nonwoven is likewise introduced.

During the consolidation process, needles pull fibers out of the nonwoven itself and form them into loops with a pillarstitch.

This web stitchbond is marketed under the name “Malivlies”, likewise by Malimo.

A general survey of the various types of mechanically consolidated fiber nonwovens can be seen in the article “Kaschierung von Autopolsterstoffen mit Faservliensen” [Lining of automobile upholstery with fiber nonwovens] by G. Schmidt, Melliand Textilberichte June 1992, pages 479 to 486.

In summary, it can be emphasized that as carrier materials all rigid and elastic sheetlike structures of synthetic and natural raw materials are suitable. Carrier materials are preferred which can be employed such that they fulfill the properties of a functional dressing. Textiles such as wovens, knitted goods, laid goods, nonwovens, laminates, nets, films, foams and papers are mentioned by way of example. These materials can further be pretreated or aftertreated. Customary pretreatments are corona discharge and hydrophobicization; familiar aftertreatments are calendering, tempering, lining, stamping and covering.

It is particularly advantageous if the carrier material is sterilizable, preferably γ-(gamma-)sterilizable.

Finally, the moisture-absorbing matrix can be covered with an adhesive-repellent carrier material, such as siliconized paper, or provided with a wound dressing or a pad.

The properties of the moisture-absorbing matrix mentioned in particular suggest use for medical products, in particular plasters, medical fastenings, wound coverings, orthopedic or phlebological bandages and dressings.

### Preparation

Silicones are processed as single- or two-component systems. Crosslinkage is carried out here as a rule as a polycondensation with elimination of acetic acid, or as a polyaddition using a platinum catalyst.

For the preparation of the matrices, a commercially available two-component system comprising polydimethylsiloxane (see figure), namely Q7-2218A+B; Dow Corning,

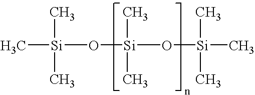

was used.

For the adjustment of the adhesive force, a polydimethylsiloxane crosslinked with silicone resin (PSA MD74602; Dow Corning) was optionally used.

The water absorption capacity of the matrix was achieved by incorporating gel-forming agents having high relative surface area in such amounts that the gel-forming agent can have intermolecular crosslinkages from the surface to the interior of the matrix. Such gel-forming agents are, for example, polyacrylic acid, polyacrylonitrile or microcrystalline cellulose. Polyacrylic acid types of the Carbopol series, Goodrich Corp., were mainly employed.

For the variation of the water absorption capacity, strong gel-forming agents having low relative surface area were additionally incorporated, such as, for example, sodium polyacrylate (Favorsorb; Stockhausen).

The preparation is carried out at room temperature in commercially available mixers. First, in the case of 2-component systems, the two silicone components are mixed with one another. After this, if required, the silicone resin component is stirred in, then the gel-forming agent(s) are incorporated and finally, if required, active compound or active compound solution is introduced.

The matrix is spread onto a carrier and the solvent of the silicone resin component is allowed to evaporate from the matrix. The length of the crosslinking reaction of the silicone matrix can be controlled temperature-dependently. The adhesive side of the matrix is covered with a separating carrier.

Active compounds to be incorporated which produce chemical disturbances during the crosslinking of the silicone matrix can be introduced as a solution via the channels of the gel-forming agents after the crosslinking reaction.

Below, in a table are indicated a number of example formulations which are particularly advantageous embodiments of the matrix.

### EXAMPLES RECIPES

### Example 1

| 23/90 | % by weight |
|---|---|
| Carbopol | 30 |
| Favorsorb | |
| Q7-2218 A | 30 |
| Q7-2218 B | 40 |
| Silicone PSA | |
| Active compound(s) | |
| Solubilizer | |

### Example 2

| 24/90 | % by weight |
|---|---|
| Carbopol | 35 |
| Favorsorb | |
| Q7-2218 A | 29 |
| Q7-2218 B | 36 |
| Silicone PSA | |
| Active compound(s) | |
| Solubilizer | |

### Example 3

| 25/90 | % by weight |
|---|---|
| Carbopol | 32 |
| Favorsorb | |
| Q7-2218 A | 31.3 |
| Q7-2218 B | 36.7 |
| Silicone PSA | |
| Active compound(s) | |
| Solubilizer | |

### Example 4

| 33/90 | % by weight |
|---|---|
| Carbopol | 30 |
| Favorsorb | |
| Q7-2218 A | 29.4 |
| Q7-2218 B | 34.4 |
| Silicone PSA | 6.2 |
| Active compound(s) | |
| Solubilizer | |

### Example 5

| 34/90 | % by weight |
|---|---|
| Carbopol | 28.2 |
| Favorsorb | |
| Q7-2218 A | 24.7 |
| Q7-2218 B | 29.4 |
| Silicone PSA | 17.7 |
| Active compound(s) | |
| Solubilizer | |

### Example 6

| 35/90 | % by weight |
|---|---|
| Carbopol | 24.6 |
| Favorsorb | |
| Q7-2218 A | 21.5 |
| Q7-2218 B | 25.6 |
| Silicone PSA | 28.3 |
| Active compound(s) | |
| Solubilizer | |

### Example 7

| 36/90 | % by weight |
|---|---|
| Carbopol | 32 |
| Favorsorb | |
| Q7-2218 A | 23.4 |
| Q7-2218 B | 27.8 |
| Silicone PSA | 16.8 |
| Active compound(s) | |
| Solubilizer | |

### Example 8

| 37/90 | % by weight |
|---|---|
| Carbopol | |
| Favorsorb | 32 |
| Q7-2218 A | 23.4 |
| Q7-2218 B | 27.8 |
| Silicone PSA | 16.8 |
| Active compound(s) | |
| Solubilizer | |

### Example 9

| 41/90 | % by weight |
|---|---|
| Carbopol | 30.8 |
| Favorsorb | 3.8 |
| Q7-2218 A | 22.5 |
| Q7-2218 B | 26.7 |
| Silicone PSA | 16.2 |
| Active compound(s) | |
| Solubilizer | |

### Example 10

| 43/90 | % by weight |
|---|---|
| Carbopol | 32 |
| Favorsorb | |
| Q7-2218 A | 23.4 |
| Q7-2218 B | 13.9 |
| Silicone PSA | 30.7 |
| Active compound(s) | |
| Solubilizer | |

### Example 11

| 44/90 | % by weight |
|---|---|
| Carbopol | 32 |
| Favorsorb | |
| Q7-2218 A | 23.4 |
| Q7-2218 B | 13.9 |
| Silicone PSA | 29.7 |
| Active compound(s) | |
| chlorhexidine diacetate | 1 |
| Solubilizer | |

### Example 12

| 48/90 | % by weight |
|---|---|
| Carbopol | |
| Favorsorb | |
| Q7-2218 A | 31.2 |
| Q7-2218 B | 46.8 |
| Silicone PSA | 17 |
| Active compound(s) | |
| ethylene glycol | 5 |
| monosalicylate | |
| Solubilizer | |

### Example 13

| 48/90 | % by weight |
|---|---|
| Carbopol | |
| Favorsorb | |
| Q7-2218 A | 32.4 |
| Q7-2218 B | 40.5 |
| Silicone PSA | 17 |
| Active compound(s) | |
| ethylene glycol | 10 |
| monosalicylate | |
| capsaicin | 0.1 |
| Solubilizer | |

### Example 14

| 50/90 | % by weight |
|---|---|
| Carbopol | |
| Favorsorb | |
| Q7-2218 A | 32.4 |
| Q7-2218 B | 40.3 |
| Silicone PSA | 17 |
| Active compound(s) | |
| ethylene glycol | 10 |
| monosalicylate | |
| capsaicin | 0.3 |
| Solubilizer | |

### Example 15

| 51/90 | % by weight |
|---|---|
| Carbopol | |
| Favorsorb | |
| Q7-2218 A | 32.4 |
| Q7-2218 B | 40.5 |
| Silicone PSA | 17 |
| Active compound(s) | |
| capsaicin | 0.3 |
| Solubilizer | |
| IPM | 10 |

### Example 16

| 53/90 | % by weight |
|---|---|
| Carbopol | 32 |
| Favorsorb | |
| Q7-2218 A | 23.4 |
| Q7-2218 B | 27.8 |
| Silicone PSA | 11.5 |
| Active compound(s) | |
| capsaicin | 0.3 |
| Solubilizer | |
| 2-octyldodecanol | 5 |

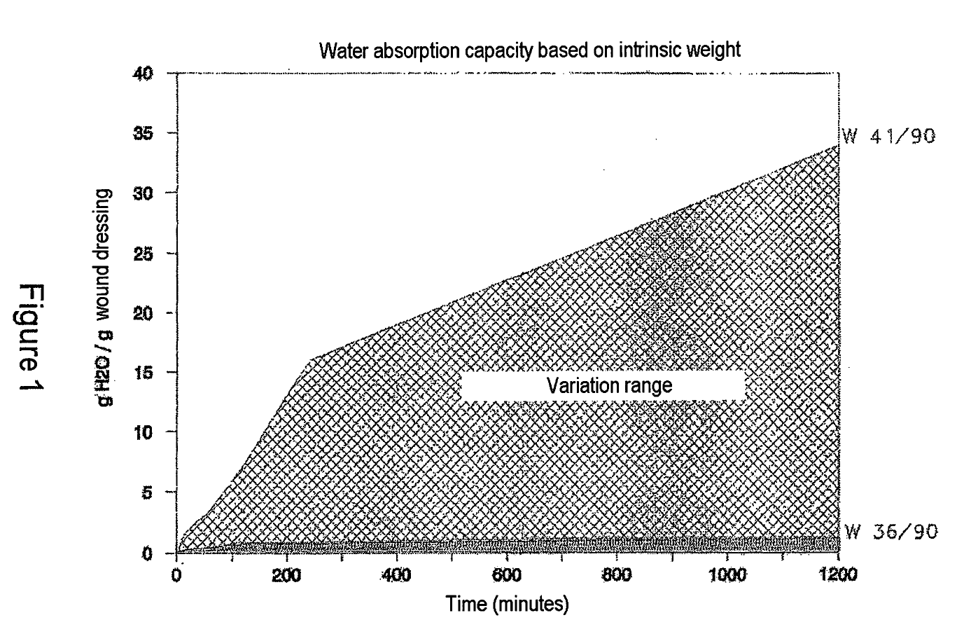

In FIG. 1, it is shown how the matrix according to the invention is outstandingly suitable as a moisture-absorbing wound dressing, namely with the aid of examples 7 (36/90) and 9 (41/90).

In detail, FIG. 1 shows by way of example the time course of the water absorption for a matrix according to the invention having a very low absorption capacity (36/90). As can be seen from the example recipe 7 accompanying it, 32% by weight of only one gel-forming agent having a high relative surface area (polyacrylic acid) were incorporated into this matrix. From this, after immersion of the sample in water at room temperature for 20 hours, a moisture absorption of 1 g of water per 1 g of matrix results.

Example recipe 9 represents, in FIG. 1, the time course for a matrix according to the invention having a high water absorption capacity (41/90). In this matrix, the proportion of gel-forming agent having a high relative surface area compared with example recipe 7 was slightly reduced to 30.8% by weight and 3.8% by weight of a gel-forming agent having a low relative surface area, but having an extremely high water absorption capacity (sodium polyacrylate), was added. From this, after immersion of the sample in water at room temperature for 20 hours, a moisture absorption of 34 g of water per 1 g of matrix results.

By selection of different gel-forming agents and variation of the proportions by weight of one or more of these gel-forming agents it is thus possible—as can be seen from FIG. 1—to set any desired moisture absorption individually in a matrix according to the invention.

Furthermore, by selection of the carrier material, depending on its water vapor permeability, the moisture content within the matrix can be set as needed during the application by evaporation of the water absorbed.

1. A silicone-based water-absorbing matrix, in particular for wound care and/or pharmaceutical/cosmetic skin treatment, the adhesive matrix consisting of
a) silicone
b) gel-forming agent
c) optionally a silicone resin.

2. The silicone-based moisture-absorbing resin as claimed in claim 1, characterized in that the matrix has the following composition:
a) silicone: 55 to 80% by weight, in particular 60 to 75% by weight
b) gel-forming agent: 20 to 40% by weight, in particular 25 to 40% by weight

3. The silicone-based moisture-absorbing matrix as claimed in claims 1 and 2, characterized in that up to 50% by weight of silicone resins are added to the matrix, in particular between 5 and 40% by weight.

4. The silicone-based moisture-absorbing matrix as claimed in at least one of the preceding claims, characterized in that the matrix preferably contains 1 to 10% by weight of ethereal oils, in particular chosen from the group consisting of eucalyptus oil, peppermint oil, camomile oil, camphor, menthol, citrus oil, cinnamon oil, oil of thyme, lavender oil, oil of cloves, tea tree oil, cajuput oil, niaouli oil, kanuka oil, manuka oil, templin oil.

5. The silicone-based moisture-absorbing matrix as claimed in at least one of the preceding claims, characterized in that superbabsorbers are added to the matrix.

6. The silicone-based moisture-absorbing matrix as claimed in at least one of the preceding claims, characterized in that the matrix preferably contains a hydrophilic filler based on cellulose and its derivatives, whose mean particle size is in the range from 20 to 60 μm.

7. The silicone-based moisture-absorbing matrix as claimed in at least one of the preceding claims, characterized in that penetration-promoting constituents in the concentration range up to 30% by weight, preferably 5 to 15% by weight, are added to the matrix.

8. The silicone-based moisture-absorbing matrix as claimed in at least one of the preceding claims, characterized in that skin-caring, cosmetic additives are added to the matrix, particularly to 0.2 to 10% by weight, very particularly to 0.5 to 5% by weight.

9. The silicone-based moisture-absorbing matrix as claimed in at least one of the preceding claims, characterized in that pharmaceutically active substances are added to the matrix, preferably up to 40% by weight, particularly to 0.1 to 25% by weight, very particularly to 0.5 to 10% by weight.

10. The silicone-based moisture-absorbing matrix as claimed in at least one of the preceding claims, characterized in that the matrix is applied to a flexible covering layer.
